(19) United States
(12) Patent Application Publication
(10) Pub. No.: US 2023/0338610 A1
(43) Pub. Date: Oct. 26, 2023

(54) DRY FOAM COMPRISING AGAR-AGAR

(71) Applicants: UNIVERSITÉ D'AIX-MARSEILLE, MARSEILLE (FR); CENTRE NATIONAL DE LA RECHERCHE SCIENTIFIQUE - CNRS, Paris (FR); ASSISTANCE PUBLIQUE HOPITAUX DE MARSEILLE, MARSEILLE (FR); TM4, PARIS (FR); UNIVERSITE PARIS-SACLAY, Gif Sur Yvette (FR)

(72) Inventors: Vincent VIDAL, LA BOUILLADISSE (FR); Raphaël HAUMONT, LINAS (FR); Thierry MARX, PARIS (FR); Benjamin GUILLET, AIX EN PROVENCE (FR)

(21) Appl. No.: 18/245,809

(22) PCT Filed: Sep. 20, 2021
(86) PCT No.: PCT/EP2021/075837

§ 371 Date: Mar. 17, 2023

(30) Foreign Application Priority Data
Sep. 18, 2020 (EP) 20306061.1

(51) Int. Cl. A61L 24/00; A61L 24/08; C08J 9/28

(52) U.S. Cl. CPC A61L 24/0036; A61L 24/08; A61L 24/0015; C08J 9/28; A61L 2430/36; A61L 2400/04; A61L 2300/44; C08J 2201/0484; C08J 2207/10; C08J 2305/12

(57) ABSTRACT

The present invention relates to a dry foam comprising agar-agar characterized by an elasticity modulus from 0.02 to 0.6 MPa, particularly from 0.15 to 0.6 MPa, more particularly from 0.3 to 0.4 MPa, a manufacturing process thereof and the uses thereof in particular as an embolization agent.

### FIELD OF THE INVENTION

The present invention relates to a dry foam comprising agar-agar and a method of making the foam. This invention further concerns the use of said foam as an embolization agent.

### BACKGROUND

Arterial embolization is a method that consists of occluding a bleeding artery, particularly in post-traumatic, oncological, and postpartum contexts. However, the embolic agents currently available are made from synthetic and/or non-ecological materials: animal pork-based animal gelatin, synthetic microparticles, metallic coils, and synthetic ethylene-based liquids (Vaidya et al. (2008); Lubarsky et al. (2009)).

In view of today's ecological and animal concerns and the renewed interest in plant-based materials, there is a need to provide new embolization agent being ecologic and of natural and non-animal origin.

The inventors have developed a dry foam comprising agar-agar with original properties that allow it to be used as an embolization agent.

### SUMMARY OF THE INVENTION

The present invention concerns a dry foam comprising agar-agar characterized by an elasticity modulus from 0.02 to 0.6 MPa, particularly from 0.15 to 0.6 MPa, more particularly from 0.3 to 0.4 MPa. Said foam may be further characterized by a maximum compressive strength from 0.008 to 0.11 MPa, particularly from 0.05 to 0.11 MPa, more particularly from 0.08 to 0.1 MPa, and/or by a density from 0.01 to 0.042 g/cm³, particularly from 0.025 to 0.042 g/cm³, more particularly from 0.03 to 0.036 g/cm³.

In addition to these previous characteristics or alternatively to these previous characteristics, the dry foam according to the invention can be defined as:
a dry foam comprising at least 95% 96%, 97%, 98%, 99% or 100% of agar-agar characterized by
a porosity, preferably an open porosity, comprised between 75% and 95% preferably between 75% and 90%, preferably between 80 and 90%, preferably between 80% to 85% or even more preferably about 82%, the porosity being measured for instance by tomography, preferably X-ray micro-tomography, followed by image analysis, for example via the ImageJ software and/or
said dry foam being non-uniform (i.e. disperse) and/or
the average pore size (i.e. diameter) of said foam is comprised between 250 microns and 700 microns preferably between 400 and 600 microns, and even more preferably about 540 microns and/or
the median pore size of said foam is comprised between 200 and 400, preferably between 200 and 300 microns and more preferably about 280 microns, and/or
said dry foam is suitable for embolization.

Said foam may further comprises a therapeutic drug, such as an anti-inflammatory drug or an anti-cancer drug, end/or may further comprises a medical imaging marker such as a radioactive isotope.

The present invention also relates to therapeutic uses of said dry foam such as the use as an embolization agent, the use in vascular embolization, the use for treating an abnormal blood flow in a blood vessel and the use in the treatment of a cancer, in particular a hepatocellular carcinoma.

### DETAILED DESCRIPTION

A first aspect of the present invention relates to a process for manufacturing a dry foam comprising agar-agar.

Agar-agar or agar is a jelly-like substance, contained in the cell wall of certain species of red algae (Rhodophyta) belonging to the families Gelidiaceae, in particular to the Gelidium and Pterocladia genus, and Gracilariaceae, in particular to the Gracilaria genus.

Agar consists of a mixture of two polysaccharides: agarose and agaropectin, with agarose making up about 70% of the mixture. Agarose is a linear polysaccharide, made up of repeating units of agarobiose, a disaccharide made up of D-galactose and 3,6-anhydro-L-galactopyranose. Agaropectin is a heterogeneous mixture of smaller molecules that occur in lesser amounts (about 30% of the mixture), and is made up of alternating units of D-galactose and L-galactose heavily modified with acidic side-groups, such as sulfate and pyruvate.

According to a particular embodiment, said process comprises the following steps:
a. Mixing agar-agar to distilled water at an amount from 1.5 to 7% (w/v), in particular from 2.5 to 7% (w/v), in particular from 3 to 7% (w/v), more particularly from 3 to 5% (w/v), even more particularly from 4 to 6% (w/v);
b. Heating between 80° C. and 100° C., for example 90° C.;
c. Cooling the mix until a temperature between 45° C. and 80° C., preferably between 50° C. and 60° C., for example 55° C.;
d. Injecting gas to obtain a foam;
e. Freezing the resulting foam between −196° C. and −40° C., in particular between −120° C. and −40° C., in particular between −80° C. and −40° C., more particularly between −60° C. and −40° C.;
f. Freeze-drying the foam to obtain a dry foam comprising agar-agar.

Preferably, step a) and step c) are performed at room temperature. In particular, at step a), agar-agar is mixed to distilled water at an amount of 1.5%, 2%, 2.5%, 3%, 3.5%, 4%, 4.5%, 5%, 5.5%, 6%, 6.5% or 7% (w/v).

The agar-agar used in step a) typically comprises from 60% to 80% of agarose and from 20% to 40% of agaropectin. In particular, the agar-agar used in step a) comprises from 65% to 75% of agarose and from 25% to 35% of agaropectin. More particularly, the agar-agar used in step a) comprises about 70% of agarose and about 30% of agaropectin.

At step d), an agar-agar foam is obtained starting from the agar-agar mix cooled at a temperature between 45° C. and 80° C., preferably between 50° C. and 60° C., for example 55° C. Preferably, the gas injected at step d) is an inert gas, medically neutral gas, i.e. non-toxic for living beings, in particular for human beings. For example, the gas may be N₂O or pressurized air. Any gas injection system can be used, such as a whipping siphon. The gas may be injected using a compressor device which introduces gas. In particular, the gas is injected between 5 and 18 bars, in particular between 5 and 10 bars, more particularly at 6 bars.

The foam obtained at step d) is recovered in a container. The container comprising the foam is then placed in the cold (step e), at a temperature between −196° C. and −40° C., in particular between −120° C. and −40° C., in particular between −80° C. and −40° C., more particularly between −60° C. and −40° C. In particular, the freezing step of the foam is performed at −40° C., −45° C., −50° C., −55° C. or −60° C. The freezing allows to block the conformation of the obtained foam. Preferably, step e) is performed immediately after step d).

The freezing time depends on the freezing temperature. The aim is that the foam be frozen to the core. For example, at a temperature of −40° C., the freezing time is around 20-30 minutes to obtain a freezing to the core. It goes without saying that the foam may be kept at cold temperature longer that the freezing time necessary to obtain a freezing to the core.

Once frozen, the foam is subjected to a freeze-drying stage (step f). The temperature of the first cycle of the freeze-drying and the start temperature of the second cycle is the same as the temperature of the previous freezing step. An agar-agar dry foam is then obtained.

In a particular embodiment, the freeze-drying starts with a first cycle at −196° C. with a fast decreasing pressure from atmospheric pressure down to about 0.0035 bar, and then a second cycle at about 0.0035 bar with a slowly and natural increasing temperature from −196° C. up to ambient temperature, and finally break the vacuum. In a particular embodiment, the freeze-drying starts with a first cycle at −40° C. with a fast decreasing pressure from atmospheric pressure down to about 0.0035 bar, and then a second cycle at about 0.0035 bar with a slowly and natural increasing temperature from −40° C. up to ambient temperature, and finally break the vacuum. In a particular embodiment, the freeze-drying starts with a first cycle at −60° C. with a fast decreasing pressure from atmospheric pressure down to about 0.0035 bar, and then a second cycle at about 0.0035 bar with a slowly and natural increasing temperature from −60° C. up to ambient temperature, and finally break the vacuum.

Optionally, an active ingredient is added to the foam after step f, for example by common impregnation techniques.

Said active ingredient may be a therapeutic drug such as an anti-inflammatory drug or an anti-cancer drug.

When the process does not comprise the addition of an active ingredient, the dry foam obtained from the process is 100% agar-agar.

According to a second aspect, the invention relates to a dry foam comprising agar-agar, obtainable by the process above described.

The dry foam comprising agar-agar according to the invention and hereby described is suitable for embolization or suitable as embolization agent. By “suitable for embolization” or “suitable as embolization agent”, it is meant that the foam has biological, morphological or physicochemical characteristics that make it usable as vascular or any passageway embolic material while ensuring ensure safety and absence of toxicity for the patient.

By “comprising agar-agar”, it is meant that the solid network (i.e. solid matrix) which constitutes the dry foam comprises at least 95% of agar-agar, in particular 95%, 96%, 97%, 98%, 99% or 100% of agar-agar. In particular, said dry foam is 100% agar-agar. Percentage is expressed by weight with respect to the foam dry weight.

The dry foam comprising agar-agar may be also called “agar-agar dry foam”.

The dry foam comprising agar-agar according to the invention is characterized by an elasticity modulus from 0.02 to 0.6 MPa, particularly from 0.15 to 0.6 MPa, more particularly from 0.3 to 0.4 MPa.

In a particular embodiment, the dry foam comprising agar-agar according to the invention is characterized by an elasticity modulus from 0.02 to 0.3 MPa.

In a particular embodiment, the dry foam comprising agar-agar according to the invention is characterized by an elasticity modulus from 0.35 to 0.6 MPa.

In a particular embodiment, the dry foam comprising agar-agar according to the invention is characterized by an elasticity modulus from 0.15 to 0.35 MPa. The elastic modulus or Young's modulus is measured, at room temperature and atmospheric pressure, by a compression on a 2×2 cm² surface of a sample of dry agar-agar foam, having a height of 1 cm, with an 8 cm diameter analysis plate. The sample is compressed from 0 to 60% of its initial height. A compression curve corresponding to the compression (σ) against the deformation (ε) is then traced. From the analysis of the initial part of the compression curve which corresponds to the elastic deformation (typically from 0 to 10% of deformation), the directing coefficient of the linear response σ=E·ε, is calculated and corresponds to the Young's Modulus (E). To perform the Young's modulus measurements, conventional compression processes were carried out, as it is already reported in literature to characterize rheological properties of a foam (Vivcharenko et al. (2020); Muhayudin et al. (2020)). In particular, the sample was stretched at a crosshead rate of 50 mm/min. A device such as a texturometer (also called texture analyzer) may be used for the compression. For example, the texturometer Almetek TA1 may be used.

The dry foam comprising agar-agar according to the invention may be further characterized by a maximum compressive strength from 0.008 to 0.11 MPa, particularly from 0.05 to 0.11 MPa, more particularly from 0.08 to 0.1 MPa.

In a particular embodiment, the dry foam comprising agar-agar according to the invention is characterized by a maximum compressive strength from 0.008 to 0.08 MPa.

In a particular embodiment, the dry foam comprising agar-agar according to the invention is characterized by a maximum compressive strength from 0.09 to 0.11 MPa.

In a particular embodiment, the dry foam comprising agar-agar according to the invention is characterized by a maximum compressive strength from 0.05 to 0.09 MPa. These maximum compressive strengths measurements are obtained for a foam deformation of 60%.

To measure the maximum compressive strength, a compression is applied with an 8 cm diameter analysis plate, on a 2×2 cm² surface of a sample of dry agar-agar foam, having a height of 1 cm, at room temperature and atmospheric pressure. The sample is compressed from 0 to 60% of its initial height. A compression curve corresponding to the compression (σ) against the deformation (ε) is then traced. The maximum compressive strength is then obtained on the curve for a 60% deformation. A compressive strength device is used, such as a texturometer (also called texture analyzer). For example, the texturometer Almetek TA1 may be used.

The dry foam comprising agar-agar according to the invention may be further characterized by a density from 0.01 to 0.042 g/cm³, particularly from 0.025 to 0.042 g/cm³, more particularly from 0.03 to 0.036 g/cm³.

In a particular embodiment, the dry foam comprising agar-agar according to the invention is characterized by a density from 0.01 to 0.03 g/cm³.

In a particular embodiment, the dry foam comprising agar-agar according to the invention is characterized by a density from 0.034 to 0.042 g/cm³.

In a particular embodiment, the dry foam comprising agar-agar according to the invention is characterized by a density from 0.025 to 0.034 g/cm³.

The mass of a dry foam sample is measured with a scale and the volume of the sample is measured for example by water displacement method. The density of the dry foam is then obtained using the following equation: density=mass of sample/volume of the dry foam sample.

In particular, when the foam is obtained by the above process starting from an aqueous mix comprising from 1.5% to 7% (w/v) of agar-agar, the elasticity modulus is from 0.02 MPa to 0.6 MPa, the maximum compressive strength is from 0.008 to 0.11 MPa, and the density is from 0.01 to 0.042 g/cm³.

When the foam is obtained by the above process starting from an aqueous mix comprising from 3% to 7% (w/v) of agar-agar, the elasticity modulus is from 0.15 MPa to 0.6 MPa, the maximum compressive strength is from 0.05 to 0.11 MPa, and the density is from 0.025 to 0.042 g/cm³.

When the foam is obtained by the above process starting from an aqueous mix comprising from 4% to 6% (w/v) of agar-agar, the elasticity modulus is from 0.3 MPa to 0.4 MPa, the maximum compressive strength is from 0.08 to 0.1 MPa, and the density is from 0.03 to 0.036 g/cm³.

When the foam is obtained by the above process starting from an aqueous mix comprising from 1.5% to 4% (w/v) of agar-agar, the elasticity modulus is from 0.02 MPa to 0.3 MPa, the maximum compressive strength is from 0.008 to 0.08 MPa, and the density is from 0.01 to 0.03 g/cm³.

When the foam is obtained by the above process starting from an aqueous mix comprising from 5% to 7% (w/v) of agar-agar, the elasticity modulus is from 0.35 MPa to 0.6 MPa, the maximum compressive strength is from 0.09 to 0.11 MPa, and the density is from 0.034 to 0.042 g/cm³.

When the foam is obtained by the above process starting from an aqueous mix comprising from 3% to 5% (w/v) of agar-agar, the elasticity modulus is from 0.15 MPa to 0.35 MPa, the maximum compressive strength is from 0.05 to 0.09 MPa, and the density is from 0.025 to 0.034 g/cm³.

When the foam is obtained by the above process starting from an aqueous mix comprising 4% (w/v) of agar-agar, the elasticity modulus is 0.3 MPa, the maximum compressive strength is 0.08 MPa, and the density is 0.03 g/cm³.

When the foam is obtained by the above process starting from an aqueous mix comprising 6% (w/v) of agar-agar, the elasticity modulus is 0.4 MPa, the maximum compressive strength is 0.1 MPa, and the density is 0.036 g/cm³.

In particular, the above elasticity modulus, maximum compressive strength and density values are obtained when using an agar-agar comprising from 60% to 80% of agarose of the mixture and from 20% to 40% of agaropectin, more particularly comprising from 65% to 75% of agarose and from 25% to 35% of agaropectin and typically with an agar-agar comprising about 70% of agarose and about 30% of agaropectin.

Table 1 below presents the Young's modulus, the maximum compressive strength for a deformation of the foam of 60% and the density of the dry foam with respect to the percentage of agar-agar used at step a. of the process.

TABLE 1
Physical characteristics of the dry foam of the invention provided by FIGS. 2, 3B and 5

| Percentage of agar-agar used in the | | Maximum compressive | |
|---|---|---|---|
| mix with distilled water in the | Young's | strength for a | |
| manufacturing process of the dry | modulus | deformation of the | Density |
| foam (w/v) | (MPa) | foam of 60% (MPa) | (g/cm³) |
| 1.5% | 0.02 | 0.008 | 0.01 |
| 3% | 0.15 | 0.05 | 0.025 |
| 4% | 0.3 | 0.08 | 0.03 |
| 5% | 0.35 | 0.09 | 0.034 |
| 6% | 0.4 | 0.1 | 0.036 |
| 7% | 0.6 | 0.11 | 0.042 |

The dry foam comprising agar-agar according to the invention may be further characterized by its porosity. Porosity can be determined according to standard techniques, such as gas expansion method or tomography such as X-ray micro-tomography. The porosity can also be measured by saturating the foam with, for instance, an organic solvent such as hexanol. It is subsequently determined the volume of the solvent incorporated in the foam. The ratio of said determined volume with regard to the total volume of the foam, represents the porosity. Porosity is therefore calculated as the ratio between the total pore volume of the foam and its total volume, and it is expressed according to the following formula: porosity=total pore volume/total foam volume.

The dry foam porosity is comprised between 75% and 95% preferably between 75% and 90%, preferably between 80 and 90%, preferably between 80% to 85%. In one embodiment the porosity is about 82%.

The foam of the invention may be further characterized in that it has an open porosity. As used herein the terms “open porosity” means the foam is essentially composed of a network of interconnected pores. In open porosity, both the solid network (i.e. solid matrix) and the pore network (i.e. the pore space) are continuous, so as to form two interpenetrating continua such as in a sponge.

The dry foam comprising agar-agar according to the invention may be further characterized by the size of the pores and the dispersion.

In one embodiment the average pore size (i.e. diameter) is comprised between 250 microns and 700 microns preferably between 400 and 600 microns, and even more preferably about 540 microns.

In one embodiment the median pore size is comprised between 200 and 400, preferably between 200 and 300 microns and more preferably about 280 microns.

In particular, the solid network (i.e. solid matrix) constitutes from 10% to 25% of the foam preferably from 15% to 20% of the foam and even more preferably about 17.6% of the foam.

In particular, the pore network (i.e. the pore space) constitutes from 75% to 90% of the foam, preferably from 80% to 85% of the foam and even more preferably about 82.4% of the foam.

In one embodiment the solid network has an average thickness comprised between 80 and 150 microns, preferably between 90 and 120 micron and more preferably about 112 microns.

As described in the experimental part, the porosity and the dispersion of the pores were measured by X-ray micro-tomography. Scans were performed on a North Star Imaging X50CT, with a XrayWorx XWT-190-TC source and a Dexela 2923 detector. The voltage was 100 kV and the amperage was 50 μA. The images were taken at a speed of 10 per second for 44 minutes. The sample used was a 2 cm*2 cm*1 cm foam cube. The data obtained by tomography were processed via the ImageJ software. Different 3D images as well as pore size and network thickness dispersions were obtained.

In one embodiment, the dry foam according to the invention is non-uniform (i.e. disperse), meaning that the pores have inconsistent size, shape and distribution within the foam. This characteristic can be assessed by tomography, such as X-ray micro-tomography.

When the foam is obtained by the above process starting from an aqueous mix comprising 4% of agar-agar (w/v), said agar-agar comprising in particular 70% of agarose and 30% of agaropectin, the average porosity is 540 microns with a median at 280 microns. The network constitutes 17.6% of the foam with an average thickness of 112 microns.

The dry foam comprising agar-agar according to the invention is further characterized by a thermal stability up to 80° C. To measure such a characteristic, a piece of dry foam (typically 2×2×1 cm³) is placed in an oven and heated at 80° C. during 24 hours.

Once cooled at ambient temperature, it is analyzed if the volume of the sample has changed. More specifically, it is analyzed if the foam is able to keep shape, or if it is partially broken or diluted.

The dry foam of the invention keeps shape at 80° C. and is then considered as being thermally stable from the ambient temperature up to 80° C. This thermal stability up to 80° C. allows the foam to perfectly tolerate a pasteurization process whose temperature rise is classically up to 65° C.-80° C. In addition, thanks to its thermal stability, the dry agar-agar foam according to the invention can be kept at temperatures up to 40-50° C. without any risk of deterioration.

Making a foam from agar-agar provides a material of vegetable origin and non-toxic for living beings. Its origin ensures its safe use in the therapeutic framework, in particular in humans. Moreover, these physical properties make it a foam with a unique texture that allows to model the dry agar-agar foam to the desired shape. The foam can be cut cleanly with precise geometrical shapes. In a particular embodiment, the modeling can be done easily with the fingers. In particular, the dry agar-agar form of the invention can be easily shaped into the shape of a torpedo suitable for example for a placement in a blood vessel by means of a catheter, such as a 4 Fr catheter as used in interventional radiology embolization techniques.

Moreover, the pore network of the dry foam according to the invention gives its exceptional liquid absorption properties. Interestingly, the foam may acts like a sponge that is to say that the foam is capable of absorbing a liquid and releasing it under the effect of compression.

The humidification of the foam according to the invention varies certain physical properties of the foam.

For example, when the dry foam comprising agar-agar has been humidified with a 0.9% saline solution, the elastic modulus is from 0.0007 to 0.011 MPa, particularly from 0.003 to 0.011 MPa, more particularly from 0.006 to 0.008 MPa.

In a particular embodiment, when the dry foam comprising agar-agar has been humidified with a 0.9% saline solution, the elastic modulus is from 0.0007 to 0.006 MPa.

In a particular embodiment, when the dry foam comprising agar-agar has been humidified with a 0.9% saline solution, the elastic modulus is from 0.007 to 0.011 MPa.

For example, when the dry foam comprising agar-agar has been humidified with a 0.9% saline solution, the maximum compressive strength is from 0.001 to 0.011 MPa, particularly from 0.004 to 0.011 MPa, more particularly from 0.008 to 0.009 MPa.

In a particular embodiment, when the dry foam comprising agar-agar has been humidified with a 0.9% saline solution, the maximum compressive strength is from 0.001 to 0.008 MPa.

In a particular embodiment, when the dry foam comprising agar-agar has been humidified with a 0.9% saline solution, the maximum compressive strength is from 0.0085 to 0.011 MPa.

These maximum compressive strengths measurements are obtained for a foam deformation of 60%.

More particularly, when a foam obtained by the above process starting from an aqueous mix comprising from 1.5% to 7% (w/v) of agar-agar, is then humidified with a 0.9% saline solution, the elasticity modulus is from 0.0007 to 0.011 MPa and the maximum compressive strength is from 0.001 to 0.011 MPa.

When a foam obtained by the above process starting from an aqueous mix comprising from 3% to 7% (w/v) of agar-agar, is then humidified with a 0.9% saline solution, the elasticity modulus is from 0.003 to 0.011 MPa and the maximum compressive strength is from 0.004 to 0.011 MPa.

When a foam obtained by the above process starting from an aqueous mix comprising from 4% to 6% (w/v) of agar-agar, is then humidified with a 0.9% saline solution, the elasticity modulus is from 0.006 to 0.008 MPa and the maximum compressive strength is from 0.008 to 0.009 MPa.

When a foam obtained by the above process starting from an aqueous mix comprising from 1.5% to 4% (w/v) of agar-agar, is then humidified with a 0.9% saline solution, the elasticity modulus is from 0.0007 to 0.006 MPa and the maximum compressive strength is from 0.001 to 0.008 MPa.

When a foam obtained by the above process starting from an aqueous mix comprising from 5% to 7% (w/v) of agar-agar, is then humidified with a 0.9% saline solution, the elasticity modulus is from 0.007 to 0.011 MPa and the maximum compressive strength is from 0.0085 to 0.011 MPa.

When a foam obtained by the above process starting from an aqueous mix comprising from 3% to 5% (w/v) of agar-agar, is then humidified with a 0.9% saline solution, the elasticity modulus is from 0.003 to 0.007 MPa and the maximum compressive strength is from 0.004 to 0.085 MPa.

When humidified with a 0.9% saline solution, the agar-agar foam according to the invention is thermally stable up to 60° C., in particular up to 50° C. From 65° C., a disintegration may be observed.

In a particular embodiment, the dry foam comprising agar-agar further comprises an active ingredient. In the present application, said active ingredient may be a therapeutic drug such as an anti-inflammatory drug or an anti-cancer drug. In these particular embodiments, the dry foam comprising agar-agar is then used as a vector to release or administrate locally a drug to a patient. The active ingredient may represent up to 5% of the foam, in particular up to 4%, up to 3%, up to 2% or up to 1%.

In a particular embodiment, the dry foam comprising agar-agar comprises a marker molecule. The marker molecule may be a medical imaging marker. Typically, a medical imaging marker is a radioactive isotope such as a radioactive isotope emitting alpha, beta or gamma photons. Such an isotope may be coupled to a substance that acts as a carrier and is thus labelled, such as fluorodeoxyglucose (FDG) labelled with fluorine18.

A third aspect of the present invention concerns the use of the dry foam comprising agar-agar above described.

In a particular embodiment, the present invention relates to the dry foam comprising agar-agar above described for use as an embolization agent.

This therapeutic embolization consists of clogging a blood vessel in order to achieve a complete occlusion with a blood flow stop. The embolization of a bleeding blood vessel, in particular an artery, may be performed for example in case of post-traumatic or postpartum contexts. In an oncological context, it is common to treat a cancer by obliterating a blood vessel, in particular an artery that supplies blood to a tumor, in order to stop the supply of blood to the tumor and thus to reduce the size of the tumor.

The capacity to absorb liquids of the dry agar-agar foam of the invention allows an in vivo use. Indeed, once introduced into the blood vessel to be obliterated, the foam will soften by absorbing body fluids and will take on the shape of the vessel to have an optimal obliteration action.

In a particular embodiment, the present invention relates to the use of a dry foam comprising agar-agar above described as an embolization agent.

In a particular embodiment, the present invention relates to the dry foam comprising agar-agar above described for use for vascular embolization.

In a particular embodiment, the present invention relates to the use of the dry foam comprising agar-agar above described for vascular embolization.

In a particular embodiment, the present invention relates to the dry foam comprising agar-agar above described for use for treating an abnormal blood flow in a blood vessel. “By “abnormal blood flow”, it is meant a blood flow which is different from what is expected in healthy patient for the same vessel. For example, an abnormal blood flow is an excessive blood flow in comparison with standard value obtained in healthy patient in the same vessel. This may be the case, when the embolization is performed on a bleeding vessel, for example in post-traumatic or postpartum contexts and/or in an increased blood flow, for example arteria venous malformations.

In a particular embodiment, the present invention relates to the use of the dry foam comprising agar-agar above described for treating an abnormal blood flow in a blood vessel.

In a particular embodiment, the present invention relates to the dry foam comprising agar-agar above described for use in the treatment of a cancer. In a particular embodiment, the present invention relates to the use of the dry foam comprising agar-agar above described in the treatment of a cancer. As an example, hepatocellular carcinoma may be treated with said dry agar-agar foam, in particular though a chemotherapy-embolization.

The present invention also related to the use of the dry foam comprising agar-agar above described as a vector for the delivery of a therapeutic drug.

In a particular embodiment, the present invention relates to an anti-cancer drug for use in the treatment of a cancer, wherein said anti-cancer drug is administered in a dry foam comprising agar-agar above described.

### FIGURES

FIG. 1: Photos of dry foams comprising agar-agar obtained from a process using an aqueous mix comprising 1.5%, 2.5%, 4%, 6% or 8% (w/v) of agar-agar.

FIG. 2: Density measurement (g·cm⁻³) of the dry agar-agar foam depending on the percentage of agar-agar (w/v) used at the step of mixing with distilled water in the manufacturing process.

FIG. 3A: Compression curve corresponding to the compression (a) (MPa) against the percentage of deformation (c). Deformation from 0 to 60% is performed on dry foams comprising agar-agar obtained from a process using an aqueous mix comprising 1.5%, 2.5%, 4%, 6% or 8% (w/v) agar-agar.

FIG. 3B: Maximum compressive strength (MPa) measured at 60% of deformation of a dry agar-agar foam depending on the percentage of agar-agar (w/v) used at the step of mixing with distilled water the manufacturing process of the foam.

FIG. 4A. Compression curve corresponding to the compression (a) (MPa) against the percentage of deformation (c). Deformation from 0 to 60% is performed on dry foams comprising agar-agar obtained from a process using an aqueous mix comprising 1.5%, 2.5%, 4%, 6% or 8% agar-agar (w/v), after being moistened with a 0.9% saline solution.

FIG. 4B. Maximum compressive strength (MPa) measured at 60% of deformation of a dry agar-agar foam after being moistened with a 0.9% saline solution, depending on the percentage of agar-agar (w/v) used at the step of mixing with distilled water in the manufacturing process of the foam.

FIG. 5: Young's modulus (E) (MPa) measurements of a dry foam comprising agar-agar depending on the percentage (%) of agar-agar (w/v) used in the manufacturing process of the foam.

FIG. 6: Young's modulus (E) (MPa) measurements of a dry foam comprising agar-agar after being moistened with a 0.9% saline solution, depending on the percentage (%) of agar-agar (w/v) used at the step of mixing with distilled water in the manufacturing process of the foam.

FIG. 7: Fatigue testing (45 cycles) of a dry agar-agar foam obtained from an aqueous mix at 4% of agar-agar (w/v), after being moistened with a 0.9% saline solution. For each cycle, the compression curve corresponding to the compression (σ) against the deformation (ε) is then traced.

FIG. 8: Pore size distribution in a dry agar-agar foam obtained from an aqueous mix at 4% of agar-agar.

FIG. 9A: 3D representation of the agar foam network characterized by micro tomography.

FIG. 9B: Cross-section of the network representing the pore size of the network.

FIG. 9C: Cross-section of the network representing the wall thickness.

FIG. 10A: Syringe (10 mL) filled sterilely by saline solution and containing the dry agar-agar foam of the invention (obtained from a water mix at 4% of agar-agar) shaped as a torpedo shaped in the syringe tip.

FIG. 10B: Syringe (10 mL) filled sterilely by saline solution and containing CuraSpon® shaped as a torpedo shaped in the syringe tip.

FIG. 11: DSA control before and after arterial embolization of left lower polar artery with a dry agar-agar foam obtained from a water mix at 4% of agar-agar.

FIG. 12: Computed tomography (CT) scan after arterial embolization of the lower left polar artery by a dry agar-agar foam obtained from a water mix at 4% of agar-agar (w/v) according to the invention, and lower right polar artery by CuraSpon®, and virtual reality reconstruction.

FIG. 13: macroscopic view (photography) of the left kidney after embolization with the foam according to the invention.

### EXAMPLE

### Material & Methods

In the present application, the ambient temperature is considered as 25° C. and the atmospheric pressure as 1013 hPa.

### 1. Manufacture of a Dry Agar-Agar Foam

Five type of dry agar-agar foams have been prepared starting from different percentage of agar-agar.

Agar-agar used comprised 70% of agarose and 30% agaropectin.

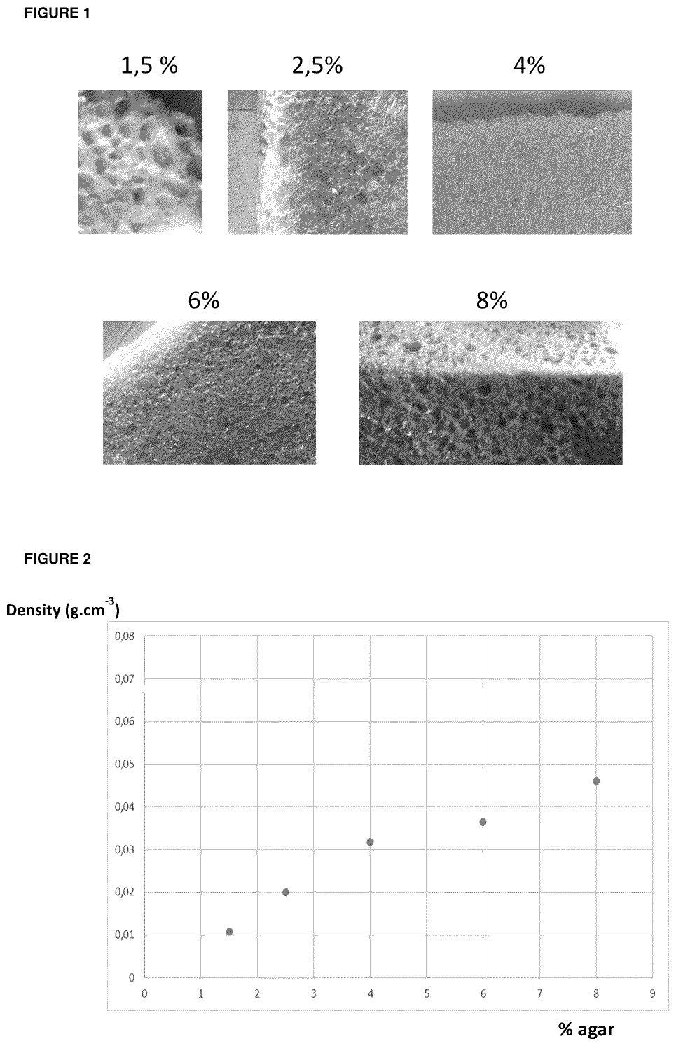

Agar-agar has been added and mixed to distilled water at an amount of 1.5, 2.5, 4, 6 or 8% (w/v). Then, the mix has been heated at 90° C. Once cooled at 55° C., the mix has been placed into a culinary siphon and N₂O gas has been injected at 6 bars to obtain a foam at ambient temperature. The foam has been recovered in a mold which has been immediately placed at −40° C. After 20-30 min, the frozen foam has been placed into a freeze-dryer. The freeze-drying starts with a first cycle at −40° C. with a fast decreasing pressure from atmospheric pressure down to about 0.0035 bar, and then a second cycle at about 0.0035 bar with a slowly and natural increasing temperature from −40° C. up to ambient temperature, and finally break the vacuum. A dry agar-agar foam has been then obtained. The texture differs depending on the agar-agar percentage used in the manufacturing process (FIG. 1). Starting with an agar-agar percentage below 3%, the dry agar-agar foam obtained is very honeycombed and elastic. With an agar-agar percentage between 4 and 6%, the foam texture is fine and elastic. However, with 8% of agar-agar, the dry foam is rigid and breakable.

### 2. Physical Characterization of the Agar-Agar Foam

2.1. Density

The density (ratio mass of sample/volume of the sample) has been measured for the dry agar-agar foams respectively obtained with 1.5, 2.5, 4, 6 or 8% of agar-agar. Results are reported in the graph at FIG. 2.

Depending on the initial percentage of agar-agar in the manufacturing process of the foam, the density of the resulting dry agar-agar foam varies greatly, approximately by a factor of 5 when going from 1.5% to 8% agar-agar.

2.2. Maximum Compressive Strength

The maximum compressive strength has been measured for the dry agar-agar foams respectively obtained from water mixes containing 1.5, 2.5, 4, 6 or 8% of agar-agar.

To measure the maximum compressive strength, a compression is applied with an 8 cm diameter analysis plate, on a 2×2 cm² surface of a sample of dry agar-agar foam, having a height of 1 cm, at room temperature and atmospheric pressure. The sample is compressed from 0 to 60% of its initial height. For the experiment, a texturometer Almetek TA1 has been used.

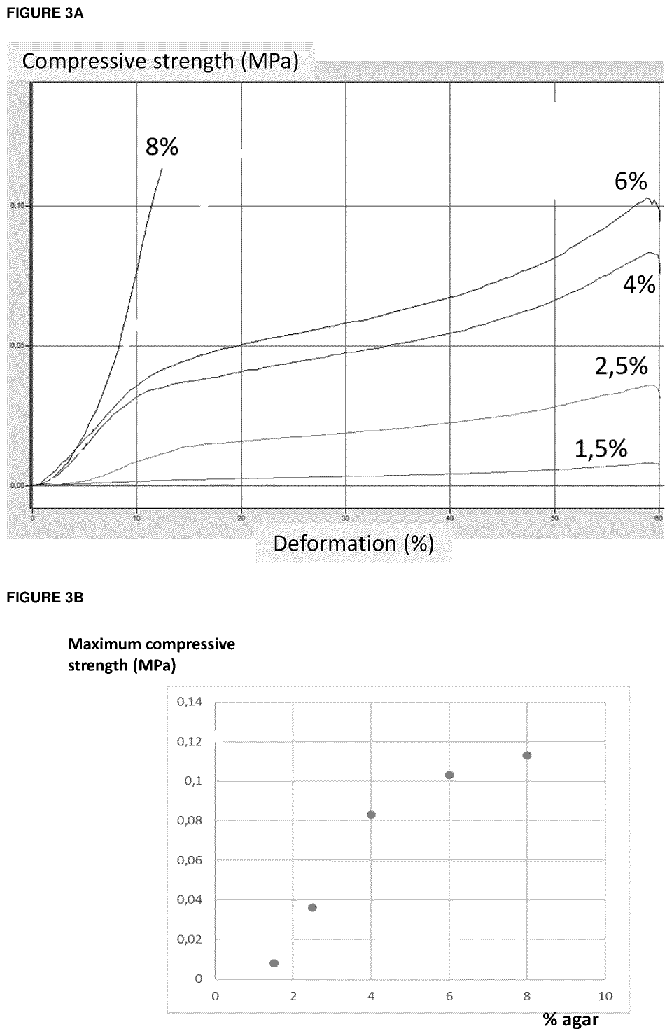

A compression curve corresponding to the compression (σ) against the deformation (ε) is then traced (FIG. 3A). The maximum compressive strength is then obtained on the curve for a 60% deformation.

Results of the maximum compressive strength for a 60% deformation depending on the percentage of agar-agar used in the manufacturing process of the foam, are reported in the graph at FIG. 3B.

Depending on the initial percentage of agar-agar in the manufacturing process, the maximum compressive strength of the resulting dry agar-agar foam varies greatly, approximately by a factor of 10 when going from 1.5% to 8% agar-agar. When the percentage of agar-agar is increased, the resulting foam has an increasingly structured and dense pore network.

When the dry agar-agar foam is obtained from a percentage of agar-agar below 2.5%, the dry foam has a weak cohesion. When it is obtained from a percentage of agar-agar between 4-6%, the dry foam has a very good cohesion. From 8% of agar-agar, the resulting agar-agar foam becomes rigid and breakable.

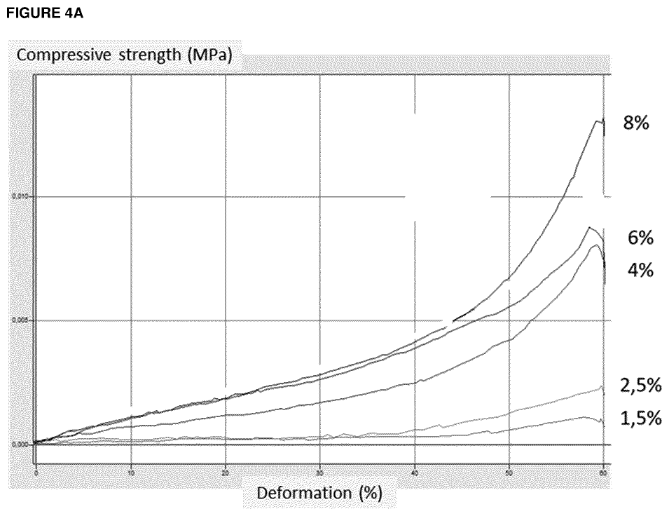

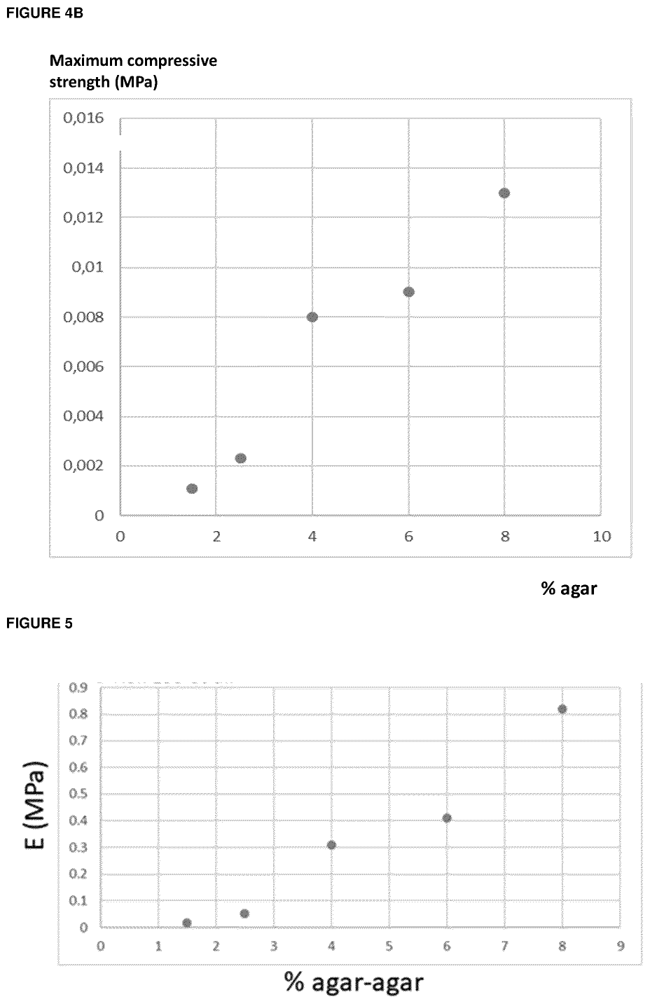

The maximum compressive strength has also been measured for the dry agar-agar foams respectively obtained with 1.5, 2.5, 4, 6 or 8% of agar-agar, after being moistened with a 0.9% saline solution. To moisten the foam, the foam is submerged into a 0.9% saline solution. The foam absorbs water almost instantly, like a sponge. The soaked foam is then placed in the texturometer. The height of the sample is measured, then the compression cycle is carried out (compression of 60% of the initial height) and the compression curve σ(ε) is traced (FIG. 4A). The maximum compressive strength is measured in the same way as for the dry foam, previously described.

Results of the maximum compressive strength for a 60% deformation measured on a humidified agar-agar foam with a 0.9% saline solution, depending on the percentage of agar-agar used in the manufacturing process of the foam, are reported in the graph at FIG. 4B.

Depending on the initial percentage of agar-agar in the manufacturing process, the maximum compressive strength of the wet agar-agar foam varies greatly, approximately by a factor of 7 when going from 1.5% to 8% agar-agar.

Interestingly, the maximum compressive strength varies by a factor 10 between the dry agar-agar foam and said foam after being moistened. This indicates that after humidification the foam is 10 times softer.

2.3. Young's Modulus

The Young's modulus or elastic modulus has been measured for the dry agar-agar foams respectively obtained from water mixes containing 1.5, 2.5, 4, 6 or 8% of agar-agar.

The Young's modulus is obtained from the compression curve previously obtained for the moistened agar-agar foam (FIG. 3A), by placing in the elastic deformation part, from 0 to 10% of deformation. The directing coefficient of the linear response σ=E·ε, is calculated and corresponds to the Young's Modulus (E).

Young's modulus measurements depending on the percentage of agar-agar used in the manufacturing process of the foam, are reported in the graph at FIG. 5.

The Young's modulus has also been measured for the dry agar-agar foams respectively obtained with 1.5, 2.5, 4, 6 or 8% of agar-agar, after being moistened with a 0.9% saline solution. The Young's modulus is obtained from the compression curve previously obtained (FIG. 4A), placing in the elastic deformation part, from 0 to 10% of deformation. The directing coefficient of the linear response σ=Eε is calculated and corresponds to the Young's Modulus (E).

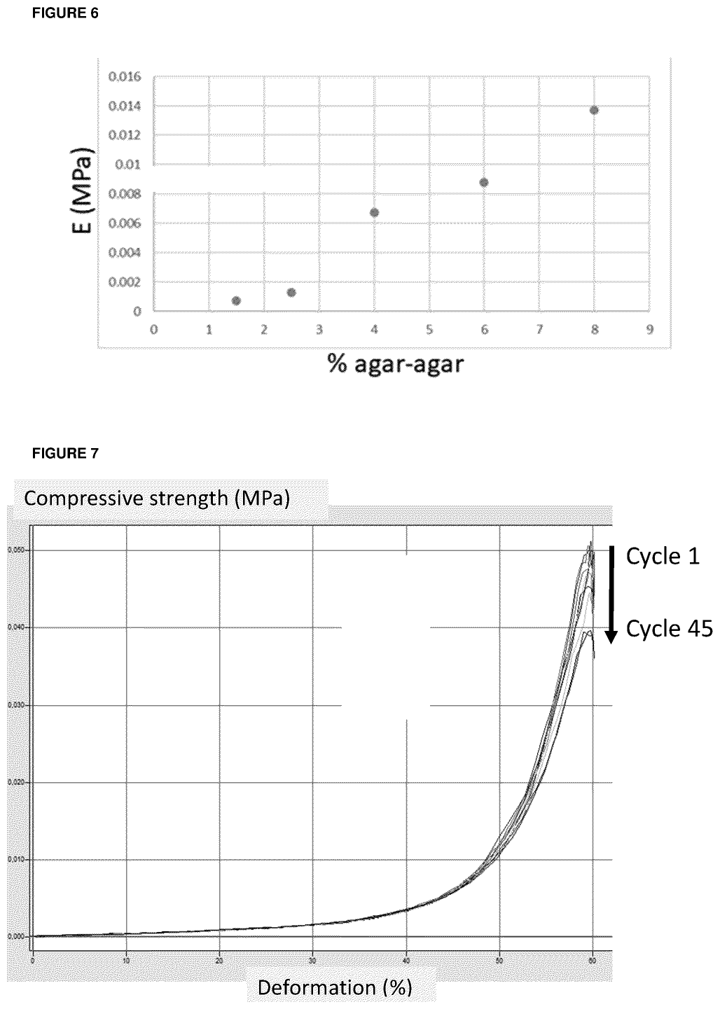

Young's modulus measurements on a humidified agar-agar foam with a 0.9% saline solution, depending on the percentage of agar-agar used in the manufacturing process of the foam, are reported in the graph at FIG. 6.

2.4. Fatigue Testing

The experiment is the same than the measurement of the maximum compressive strength, except that after compression, the foam is let relax, and a new compression is applied.

The fatigue testing has been carried out on a dry agar-agar foam obtained with 4% of agar-agar, after being moistened with a 0.9% saline solution, in order to investigate the “sponge” effect.

A compression is applied with an 8 cm diameter analysis plate, on a 2×2 cm² surface of a sample of dry agar-agar foam, having a height of 1 cm, at room temperature and atmospheric pressure. The sample is compressed from 0 to 60% of its initial height and let relax. The cycle of compression/relaxation is repeated 45 times on the same sample. For the experiment, a texturometer Almetek TA1 has been used.

A compression curve corresponding to the compression (σ) against the deformation (ε) is then traced for each cycle (FIG. 7).

After 45 cycles of compression/relaxation, the structure is preserved under high mechanical stress. A slight loss of maximum compressive strength is observed after 40 cycles. The reversibility of the foam over 40 compression/relaxation cycles under wet conditions reflects this so-called “sponge” effect.

2.5. Foam Microstructure

X-ray micro-tomography allows to observe the microstructure of the foam, i.e. its porosity and the dispersion of its pores.

Scans have been performed on a North Star Imaging X50CT, with a XrayWorx XWT-190-TC source and a Dexela 2923 detector. The voltage was 100 kV and the amperage was 50 μA. The images have been taken at a speed of 10 per second for 44 minutes. The data obtained by tomography are processed via the ImageJ software. Different 3D images as well as pore size and network thickness dispersions are obtained.

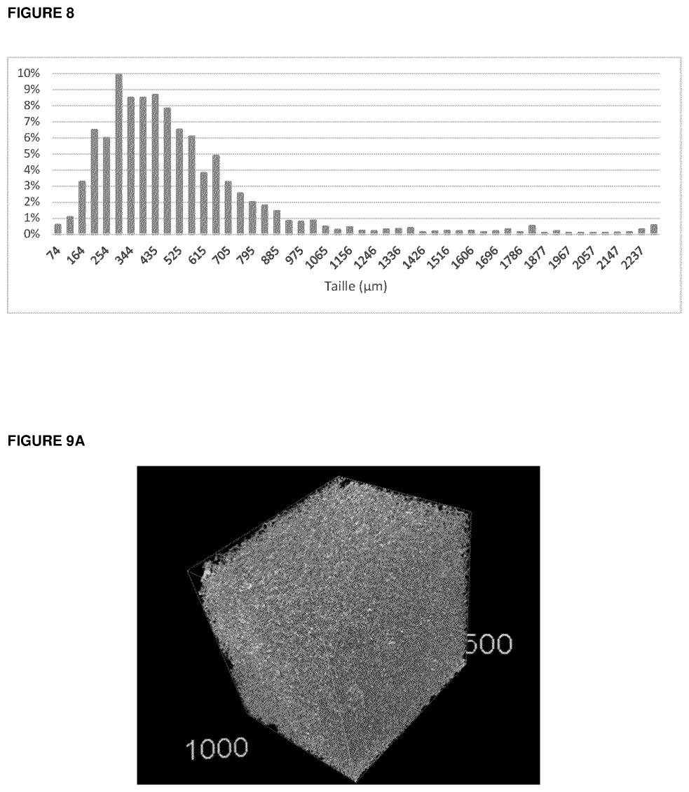

Results at FIG. 8 have been obtained from a dry agar-agar foam obtained with 4% of agar-agar, said agar-agar comprising 70% of agarose and 30% of agaropectin, on a cubical foam sample of 2 cm*2 cm*1 cm.

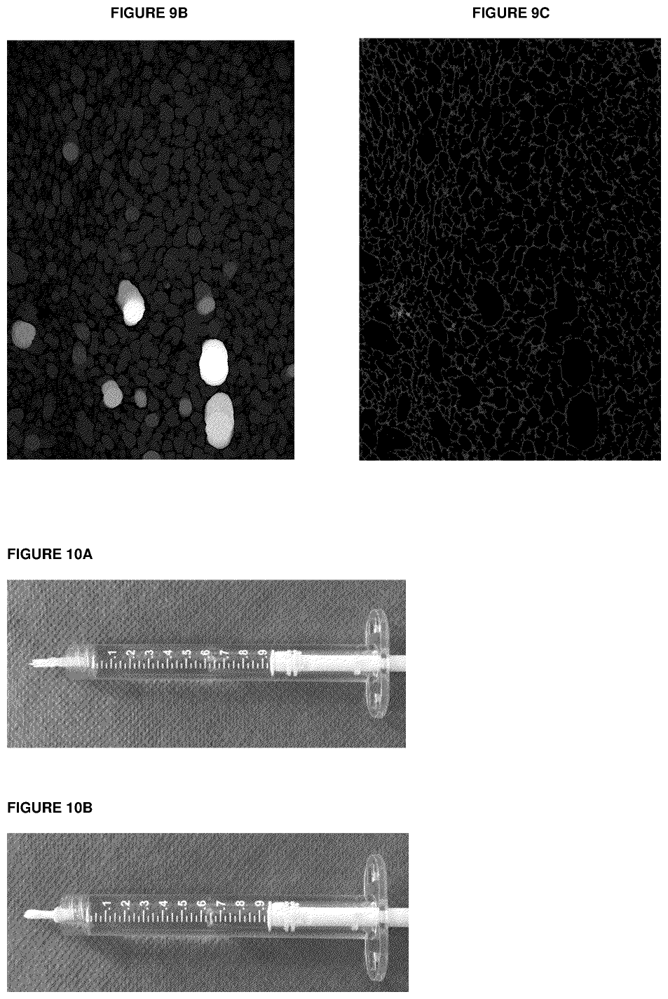

The network constitutes 17.6% of the foam with an average thickness of 112 microns. The average pore size is 540 microns with a median of 280 microns, indicating the presence of very large bubbles visible in the processed images (FIGS. 9B and 9C).

Images at FIGS. 9A, 9B and 9C show the details of the network of a dry agar-agar foam obtained from a 4% of agar-agar mix. The pores size is very heterogeneous and the distribution of pores of different sizes is also very heterogeneous. This polydispersity shows that the foam of the invention can be compared to a natural sponge. Indeed, the heterogeneity of the size of the pores allows a fast absorption of water and a swelling, while having a low density and a resistance to strong pressures. As it can be seen, the foam has an open porosity (i.e. the foam is essentially composed of a network of connected pores).

### 3. Therapeutic Use of the Dry Agar-Agar Foam as Embolization Agent

3.1. Animal Settings and Embolization Procedure

A dry agar-agar foam obtained from the manufacturing process of the invention starting with a water solution at 4% of agar-agar, has been tested in vivo on swine as an embolization agent. CuraSpon®, an embolization agent made of pork gelatin, usually used in clinical practice for transient embolization, has been used as a comparative. The swine has been used as an embolization model because its renal arteries are easy to catheterize. Besides, its anatomy is similar to that of human (with an inferior polar artery and a superior polar artery that can be clearly identified), and the embolization of only one polar artery per kidney remains compatible with life for the 3 months study duration. In addition, double and symmetrical organs such as the renal model also allows having both target and control areas in each kidney while comparing the efficacy test and control agent between the contralateral kidneys.

Four Petrain swine (30 kg, 6 months old) were used in the study. To anaesthetize the swine during the application of the embolization agent, following intramuscular sedation (20 mg/kg ketamine and 0.03 mg/kg acepromazine), the animals were placed in a dorsal, recumbent position. A venous catheter was inserted into a large ear vein for blood sampling and intravenous access. Induction of anesthesia was obtained by 2 mg/kg propofol. After orotracheal intubation, anesthesia was maintained with gaseous sevoflurane (2%) by mechanical respiration (Dräger Zeus®, Dräger Inc., Telford, PA, US). Aseptic techniques were used throughout the procedure.

A digital subtraction angiography system (DSA) (Fluorostar, General Electric Medical System, Minneapolis, MN, USA) was used for endovascular procedures. Aseptic percutaneous access was performed by femoral arterial puncture under ultrasound guidance with the placement of a 6 French (F) vascular introducer by the Seldinger method. Catheterization of arterial vascular targets was performed by an experienced interventional radiologist using a Cobra Wirebraid 5F catheter (Cordis, Fremont, California, USA). Embolization was performed once the catheter was placed within each target artery (polar kidney artery).

The embolization agent, i.e. the agar-agar dry foam according to the invention or CuraSpon®, was shaped in torpedo and placed on tip of syringes containing a saline solution (FIGS. 10A and 10B). The agar-agar dry foam according to the invention was easily introduced into the catheter and then flushed with saline. No adhesion or blockage of the catheter has been noticed.

Two swine were embolized as acute setting. The dry agar-agar foam according to the invention was tested in torpedo form in different vascular territories (kidney, lumbar and mesenteric arteries). This scheme of embolization was performed to evaluate the ease, reproducibility and feasibility of foam of the invention torpedo manipulation as an embolic agent and releasing through the catheter. The effectiveness (arterial occlusion success) and the safety (risk of off-target embolization) were also examined.

Two swine were embolized in chronic setting, in which embolization with the dry agar-agar foam according to the invention, in the form of a torpedo, was performed on the left side kidney, whereas the right-side embolization was performed with torpedoes of CuraSpon®, as a comparison. Embolization with torpedoes respectively with the dry agar-agar foam according to the invention and CuraSpon® were realized once the catheter was placed within each target artery. The embolization was performed in the upper or lower polar branch of each kidney depending on the easy-catheterization access.

The two animals were followed during each endovascular procedure with a bi-daily clinical valuation. Repeated angiographic evaluations (DSA) were also realized to evaluate artery occlusion effectiveness and possible off-target embolization. The efficacy and short- to medium-term (3 months) tolerance of the dry agar-agar foam and CuraSpon® embolizations were assessed for each animal.

3.2. Primary Endpoint and Short-Term Evaluation

The primary endpoint occurred when complete artery occlusion was achieved, or by obtaining significant reflux in the main renal artery. The percentage of embolized extent was estimated by subtracting the contoured renal surface before and after embolization (Horos™ Software, v3.3.6).

Short-term secondaries endpoints were safety (no off-target embolization), ease and duration of the the dry agar-agar foam embolization procedure compared to that of Curaspon®. This duration was established as the time between the last DSA acquisition before the start of the embolic agent administration, and the final DSA control after having obtained the main endpoint (target artery occlusion). The off-target embolized tissue area, when there was one, was estimated with the same method of the calculation of angiogram defect detailed previously by DSA subtraction method.

3.3. Medium-Term Evaluation: Clinical Criteria, CT and DSA Controls, Histology Study

The 3-month (M3) follow-up was chosen in accordance with reference standard for animal studies ISO10993-6 in order to study the medium-term effects of implanted products.

After embolization procedure and during the 3 months follow-up, the four swine underwent a bi-daily clinical evaluation, especially to look for signs of infection or inflammation (temperature abnormalities, anorexia, loss of weight and signs of undernutrition, abnormal behavior).

Computed tomography (CT) explorations were performed one day before embolization (pre-embolization control), at M1 and at M3 after the procedure. Medium-term CT controls evaluated arterial occlusion, possible complications (urinoma, arterial aneurysm, signs of infection: infiltration of peri-renal fat, abscess, local or distant lymphadenopathy), cortical thickness and each kidney's volume (IntelliSpace Portal, Philips, 2015). The volume measure was estimated by repetitive contouring method. The decrease of kidney's volume between before and after embolization was evaluated for each group.

DSA controls at M3 assessed the persistence of the arterial occlusion, and the residual angiogram defect as detailed for short-term endpoints.

After this 3-month follow-up period, animals were euthanized in order to perform histological analyzes on explanted kidneys after sacrifice. Kidneys were harvested after careful dissection, and then both medullary and cortical parts were immersed in buffered 4% formalin liquid fixation. Samples were cut (4 μm thick) from paraffin blocks (Microm, France) and examined using hematoxylin-eosin (HE) staining (AutoStainer, DRS 2000 Saqura). The following parameters were evaluated: fibrin, neovascularization, hemorrhage, revascularization, cellular inflammatory parameters (fibroblasts, polynuclear, lymphocytes, macrophages, giant cells) and acute tubular necrosis.

3.4. Results

Each target artery was catheterized according to standard procedure without technical failure. Embolization was feasible in each territory and without any general complication during the different procedures.

Embolization was performed for each target artery, without any difficulty, demonstrated by non-opacification in downstream arteries. There was none off-target embolization. The bi-daily clinical examination about the 2 swine in chronic setting did not show any sign of significant deterioration of general condition, or infection: the animals showed no significant weight loss, no sign of anorexia, no intrarectal temperature abnormality.

All vascular targets were effectively occluded, both for the acute and chronic models. DSA controls at D1, D3, D8, M1 and M3 showed persistence occlusion for kidneys arteries embolized with the dry agar-agar foam of the invention. Comparatively, the arteries initially occluded by the Curaspon® were permeable on D8 control and on all the following controls. Thus, 8 days after embolization, Curaspon® is reabsorbed. This confirms that the Curaspon® is not suitable for obliterating an artery over one week and may be only used for transient embolization.

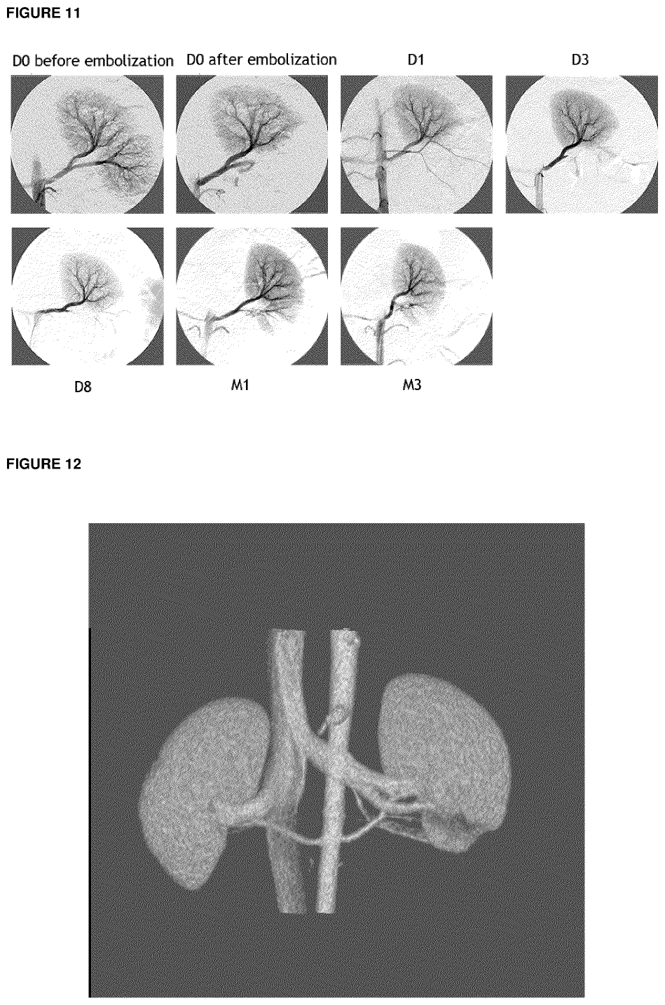

CT controls at M1 and M3 (FIG. 11) showed cortical atrophy corresponding to the embolized area. For kidneys embolized with the dry agar-agar foam of the invention, a significant decrease of the volume (atrophy) was observed. The average kidney's volume embolized by Curaspon® showed no significant decrease of the volume in comparison of the kidney's volume before embolization. There were no CT signs of complication (abscess, local lymph nodes, peri-renal infiltration, urinoma or renal excretion failure) on each control. This demonstrates an efficient and stable embolization over time with the dry agar-agar foam.

Similarly, computed tomography (CT) scan after arterial embolization of the lower left polar artery with the foam of the invention, and lower right polar artery by CuraSpon®, associated with virtual reality reconstruction (FIG. 12) shows on a more macroscopic level a significant atrophy of the lower left kidney whereas the right kidney embolized with Curaspon® shows no significant decrease of its volume.

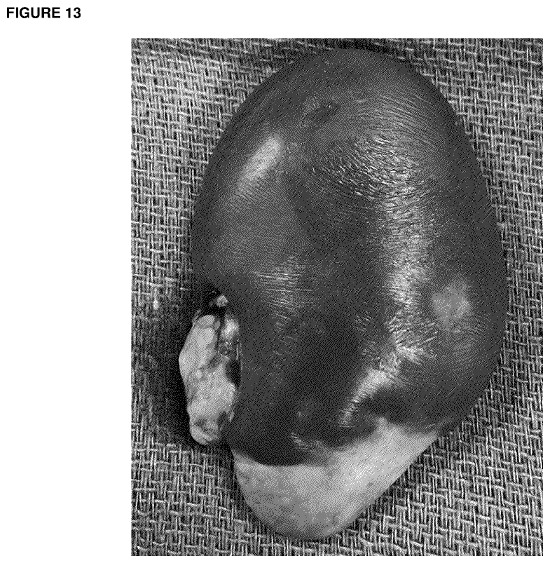

As can be seen of the photography of the explanted kidney embolized with the foam of the invention (FIG. 13), an atrophy of the inferior part of the kidney is visible by color change. The non-embolized part of the kidney remains unchanged and in good working order.

The end-of-follow-up histological analysis at M3 did not show any sign of significant tissue inflammation (data not shown). Histological analysis at M3 showed no significant difference of necrosis inflammatory parameters between the two groups, especially no sign of abscess or granuloma. Necrotic rearrangements were found for both groups, associating calcifications, micro-cysts with thin walls, and also non-specific hemorrhagic changes supposed to be of post necrosis origin or related to manipulations during harvesting. The dry agar-agar foam of the invention is then safe and non-toxic for an in vivo use.

3.5. Conclusion

These in vivo results obtained on swine constitute a proof of concept of a new embolization procedure, using biological embolic agent, made from vegetable agar-agar foam. It demonstrates the efficacy and safety of this new agent. This study demonstrates that the dry agar-agar foam of the invention may be used as a permanent embolization agent and can be easily shaped in a torpedo shape like porcine-gelatine Curaspon®.

### BIBLIOGRAPHY

Sandeep Vaidya, M.D.,1 Kathleen R. Tozer, M.D.,1 and Jarvis Chen, M.D.1 An Overview of Embolic Agents. Semin Intervent Radiol. 2008 September; 25(3): 204-215;
Lubarsky M, Ray C E and Funaki B 2009 Embolization agents—which one should be used ? Semin. Intervent. Radiol. 26 352-7;
V. Vivcharenko, A. Benko et al., Elastic and biodegradable chitosan/agarose film revealing slightly acidic pH for potential applications in regenerative medicine as artificial skin graft. International Journal of Biological Macromolecules. Volume 164, 1 Dec. 2020, Pages 172-183;
N. A. Muhayudin, K. S. Basaruddin, et al., Evaluating compressive properties and morphology of expandable polyurethane foam for use in a synthetic paediatric spine. Journal of Materials Research and Technology. Volume 9, Issue 2, March-April 2020, Pages 2590-2597.

1. Dry foam comprising at least 95% of agar-agar suitable for embolization characterized by an elasticity modulus from 0.02 to 0.6 MPa.

2. Dry foam comprising agar-agar according to claim 1 further characterized by a maximum compressive strength from 0.008 to 0.11 MPa.

3. Dry foam comprising agar-agar according to claim 1 further characterized by a density from 0.01 to 0.042 g/cm³.

4. Dry foam comprising agar-agar according to claim 1 having a porosity comprised between 75% and 95%.

5. Dry foam comprising agar-agar according to claim 4 having a porosity comprised between 80% to 85%.

6. Dry foam comprising agar-agar according to claim 4 wherein the foam has an open porosity.

7. Dry foam comprising agar-agar according to claim 1 wherein said dry foam is non-uniform.

8. Dry foam comprising agar-agar according to claim 1 having an average pore size comprised between 250 microns and 700 microns, preferably between 400 and 600 microns, and even more preferably about 540 microns, and having a median pore size comprised between 200 and 400, preferably between 200 and 300 microns and more preferably about 280 microns.

9. Dry foam comprising agar-agar according to claim 1, further comprising a therapeutic drug.

10. Dry foam comprising agar-agar according to claim 9, wherein said therapeutic drug is an anti-inflammatory drug or an anti-cancer drug.

11. Dry foam comprising agar-agar according to claim 1, wherein said dry foam comprises a medical imaging marker.

12. Dry foam comprising agar-agar according to claim 11, wherein said medical imaging marker is a radioactive isotope.

13. (canceled)

14. A method of conducting vascular embolization in a subject in need thereof, comprising
introducing the dry foam comprising agar-agar of claim 1 into a blood vessel of the subject so as to occlude the blood vessel and stop blood flow.

15. The method of claim 14, wherein the blood vessel has an abnormal blood flow.

16. The method of claim 14, wherein the blood vessel supplies blood to a cancer.

17. The method of claim 16, wherein the dry agar-agar comprises an anti-cancer drug.

18. Process for manufacturing a dry foam comprising agar-agar according to claim 1, comprising the following steps:
a. mixing agar-agar to distilled water at an amount from 1.5 to 7% (w/v);
b. heating between 80° C. and 100° C.;
c. cooling the mix until a temperature between 45° C. and 80° C.;
d. injecting gas to obtain a foam;
e. freezing the resulting foam between −120° C. and 40° C.;
f. freeze-drying the foam to obtain the dry foam comprising agar-agar.
